(19) United States
(12) Patent Application Publication
(10) Pub. No.: US 2015/0285785 A1
(43) Pub. Date: Oct. 8, 2015

(54) MONITORING AND/OR CHARACTERISING BIOLOGICAL OR CHEMICAL MATERIAL

(71) Applicant: The University of Nottingham, Nottingham (GB)

(72) Inventors: Mike Somekh, Nottingham (GB); Melissa Mather, Nottingham (GB); Jing Zhang, Nottingham (GB)

(73) Assignee: The University of Nottingham, Nottingham (GB)

(21) Appl. No.: 14/425,844

(22) PCT Filed: Sep. 4, 2013
(86) PCT No.: PCT/GB2013/052315

§ 371 Date: Mar. 4, 2015

(30) Foreign Application Priority Data
Sep. 4, 2012 (GB) 1215771.5

(51) Int. Cl. G01N 33/50; G02B 21/36; G01N 21/55

(52) U.S. Cl. CPC G01N 33/5008; G01N 21/55; G02B 21/365; G01N 2201/12

(57) ABSTRACT

A method of monitoring and/or characterising biological or chemical material arranged on a substrate comprises: • producing a first image of the biological or chemical material using total internal reflection microscopy (TIRM); and • producing a second image of the biological or chemical material using transmitted or reflected light microscopy.

The present invention relates to a method of monitoring and/or characterising biological or chemical material, e.g. cells. In particular, the invention relates to a method of monitoring and/or characterising cells in vitro. The invention also relates to an apparatus for carrying out the method.

Regenerative medicine, in particular cell therapy, has great potential for treating many illnesses, diseases and conditions. The research and development of new cell therapies requires cells to be grown or cultured. Ever larger scales of manufacture or production of cells may be required, in order to ensure that high quality regenerative medicine products can be produced consistently at an economically acceptable price.

It is necessary to monitor and/or characterise the cells that have been grown or cultured.

Currently, cell populations are routinely monitored to assess quality using conventional biological analysis, e.g. cell surface markers, gene expression. This approach is destructive, not suitable for in process measurements and renders time course experiments impossible.

Alternatively, non-destructive approaches that assess cell morphology can be used, with light microscopy techniques, e.g. bright field or phase contrast imaging, being the primary methods. These microscopy techniques can sometimes be combined with the use of exogenous labels such as fluorescent markers. This can provide functional information, but has the disadvantage that such cell modifications are invasive and potentially toxic to the cells.

A first aspect of the invention provides a method of monitoring and/or characterising biological or chemical material arranged on a substrate comprising:
producing a first image of the biological or chemical material using total internal reflection microscopy (TIRM); and
producing a second image of the biological or chemical material using transmitted or reflected light microscopy.

In an embodiment, the biological or chemical material may comprise one or more bacteria or cells. The cell(s) may be any type of cell. For instance, the cell(s) may be plant cells or mammalian cells. The cell(s) may be stem cells. In an embodiment, the cell(s) may have been grown, cultured, produced or manufactured for use in regenerative medicine, e.g. cell therapy.

The biological or chemical material may comprise a colloid.

In general, the method may be used to monitor and/or characterise any biological or chemical material that will adhere at least partially to the substrate.

In an embodiment, the cell(s) may be provided in a culture comprising the cell(s) and a growth medium. The cell(s) may be provided in a monolayer culture. One or more cells may be monitored and/or characterised at a time.

Total internal reflection microscopy (TIRM) is a non-fluorescent imaging technique which is based on the principle that an object with refractive index (n₃) will scatter an evanescent wave created when a light beam undergoes total internal reflection at an interface between two media with different refractive indices, such as glass (n₁) and air (n₂), where n₃>n₂.

The object with refractive index n₃, which may be a cell or at least a portion thereof, within the evanescent field, frustrates the total internal reflection. Typically, an image results with dark regions indicating the object in the evanescent field against a light background.

The evanescent wave does not propagate; it decays exponentially with distance. Accordingly, the evanescent wave has only a small penetration depth. The penetration depth may be up to 200 nm, e.g. around 150 nm. Therefore, a very thin region of a sample located directly above the substrate is illuminated.

Typically, a high numerical aperture objective lens is used in TIRM. The objective lens may have a numerical aperture of 1.4 or more, e.g. 1.49 or more.

TIRM can provide high contrast, low artefact images.

The second image may be produced using phase contrast illumination, bright field illumination, cross-polarized light illumination or dark field illumination.

In an embodiment, the first image and the second image may be produced substantially simultaneously.

In an embodiment, the first image and the second image may be produced at different times.

In an embodiment, the first image and the second image may be of substantially the same portion of the biological or chemical material.

The information provided by the reflected or transmitted light microscopy complements the information provided by TIRM and vice versa.

In an embodiment, the method may further comprise processing and/or analysing the first image and the second image, e.g. automatically processing and/or analysing the first image and the second image using software.

Processing and/or analysing the first image and the second image may comprise comparing the first image with the second image.

Processing and/or analysing the first image and the second image may involve segmenting the first image and/or the second image, in order to identify an area of the biological or chemical material, e.g. cells.

TIRM can provide information on the adhesion of the biological or chemical material to the substrate, because only a very thin region of a sample located directly above the substrate is illuminated. Transmitted or reflected light microscopy cannot provide such information.

In an embodiment, the image processing may generate a parameter or metric. The parameter or metric may provide a means of quickly and usefully characterising the chemical or biological material.

In an embodiment, processing and/or analysing the first image and the second image may comprise, additionally or alternatively, comparing one or more metrics or parameters derived from the first image and the second image.

Cell adhesion is thought to be tightly linked to cell function and morphology. Accordingly, assessing cell adhesion may provide useful information on cell function and morphology.

In an embodiment, cell adhesion may be assessed by comparing the first image with the second image.

In an embodiment, the parameter generated by the image processing and/or analysis may comprise a ratio of the surface area of the biological or chemical material that appears to be adhered to the substrate to the overall surface area or cross section of the biological or chemical material.

Advantageously, generating a parameter or metric may allow for quick and useful characterisation and/or monitoring of the chemical or biological material.

The method may be carried out automatically, thereby providing increased repeatability by reducing dependence on the skill of the worker carrying out the assessment. Measurements may also be carried out relatively quickly. Advantageously, cell monitoring and/or characterisation and/or classification may be carried out automatically.

In an embodiment, the chemical or biological material arranged on the substrate may be located within a chamber, e.g. an incubator, operable to provide a controlled environment. For instance, the temperature within the chamber may be held at a particular temperature, typically 37° C. when characterising and/or monitoring cells, and/or the content of the atmosphere within the chamber may be controlled.

In an embodiment, the method may be carried out over a period of time, in order to monitor the chemical or biological material over the period of time. The method may be carried out continuously over, or at intervals during, the period of time. The period of time may be of the order of days, weeks or even longer. Therefore, the first image and the second image may be produced as snapshots from a continuous recording.

For instance, the method may be used to monitor cell differentiation or the response of cells or bacteria to a stimulus such as a chemical agent, a disease, a drug or a change in conditions.

Advantageously, the method may provide label-free analysis. Conveniently, this means that live cells can be characterised and/or monitored without being harmed. The cells can be returned to their population after monitoring and/or characterisation and then re-used. Accordingly, loss of cells from a cell population during monitoring and/or characterisation may be minimised or even eliminated.

Advantageously, the method may provide live cell imaging, typically performed with two imaging channels or modalities (TIRM and reflected or transmitted light microscopy) with two fields of view.

The method may provide time-lapse imaging.

The TIRM and reflected or transmitted light microscopy channels or modalities may be operated simultaneously or sequentially, e.g. alternately, in order to produce the first image and the second image.

Advantageously, high resolution images may be produced, as a result of the use of a TIRM objective lens having a high numerical aperture, e.g. a numerical aperture of 1.4 or more.

Typically, the highest lateral resolution achievable may be of the order of 250 nm. In the z-direction, changes in position of the biological or chemical material, e.g. changes in position of a cell membrane, much less than the penetration depth of the evanescent waves may be detected. For instance, changes in position of the order of 100 nm may be detected.

In an embodiment, the method may comprise obtaining a stack of images in the z-plane, in order to overcome the effects of focal drift. The stack of images may be obtained automatically, e.g. in accordance with a program. The image(s) within the stack of images that is/are most in focus may be determined and selected automatically using image processing software.

Advantageously, the method may allow for information to be obtained in real-time.

Accordingly, the method may be carried out as a step in a cell manufacturing process.

A second aspect of the invention comprises a microscope for monitoring and/or characterising biological or chemical material on a substrate comprising:
a first optical system for viewing and/or imaging the biological or chemical material using total internal reflection microscopy; and
a second optical system for viewing and/or imaging the biological or chemical material using reflected or transmitted light microscopy;
   wherein the first optical system and the second optical system share an objective lens.

In an embodiment, the shared objective lens may have a high numerical aperture, e.g. a numerical aperture of 1.4 or more.

In an embodiment, the shared objective lens may provide a magnification of at least 20×. For instance, the shared objective lens may provide a magnification of 40×, 60×, 80× or 100×.

In an embodiment, the first optical system and/or the second optical system may comprise a source of light, which may comprise a light emitting diode (LED). Each source of light may be a monochromatic source of light. Where each optical system has a monochromatic source of light, the monochromatic sources of light may each emit a different colour, e.g. red and blue.

The first optical system and/or the second optical system may be provided with one or more image capture devices, e.g. charge-couple device (CCD) cameras. For instance, the first optical system and/or the second optical system may be provided with a plurality of image capture devices, each image capture device being configured to capture a different field of view. Accordingly, images of more than one field of view may be captured simultaneously.

In an embodiment, the microscope may comprise image processing and/or analysis means, operable to process and/or analyse images produced by the first optical system and/or the second optical system.

In an embodiment, the microscope may comprise a spatial light modulator. The spatial light modulator may enable phase stepping and programmable selection of bright field or phase contrast imaging. In addition, the actual light phase may be determined, from which the topography of an imaged object, e.g. a cell, can be reconstructed.

In an embodiment, the objective lens may be a bright field of a phase contrast objective lens. When a bright field objective lens is used the transmitted or reflected light microscopy mode of imaging may use a ring of illumination and a fixed or a programmable external phase plate.

In an embodiment, the microscope may be part of or attached or attachable to an apparatus for manufacturing or producing cells, e.g. for use in regenerative medicine.

In an embodiment, the microscope may be portable and/or may be an add-on for an existing commercial microscope.

A third aspect of the invention provides a method of manufacture of biological material, e.g. cells, comprising:
culturing or growing the biological material;
characterising and/or monitoring the biological material by a method according to the first aspect of the invention; and/or
using a microscope according to the second aspect of the invention to characterise and/or monitor the biological material.

In order that the invention may be well understood, it will now be described, by way of example only, with reference to the accompanying drawings, in which:

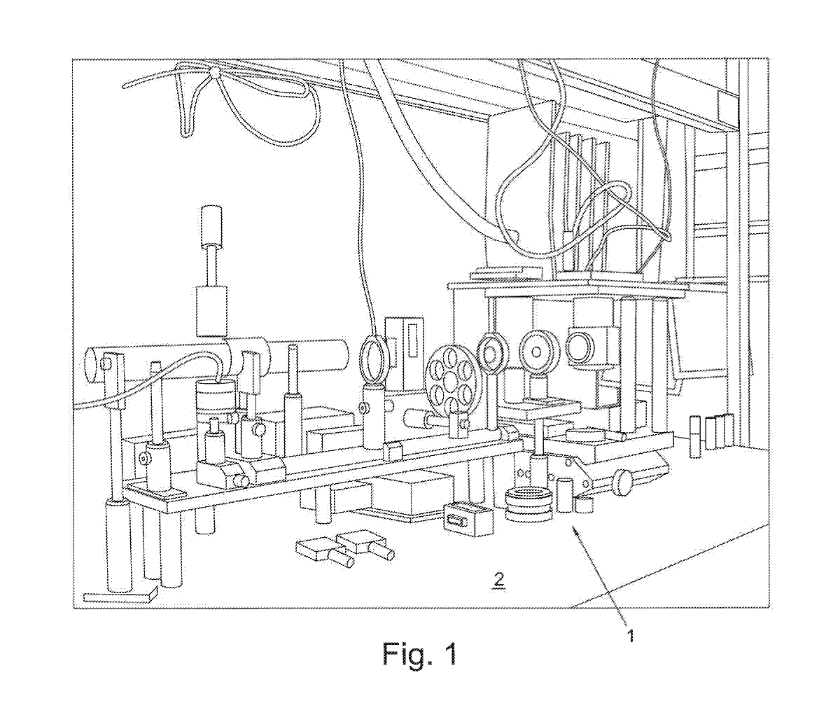

FIG. 1 is a photograph of an embodiment of a microscope according to the invention;

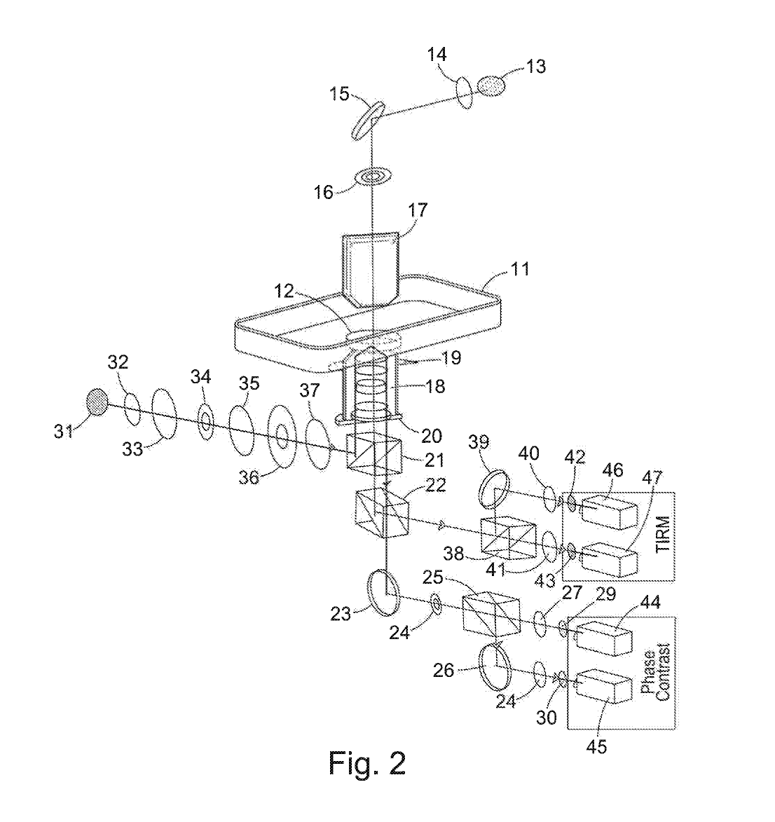

FIG. 2 is a schematic diagram illustrating how the microscope shown in FIG. 1 works;

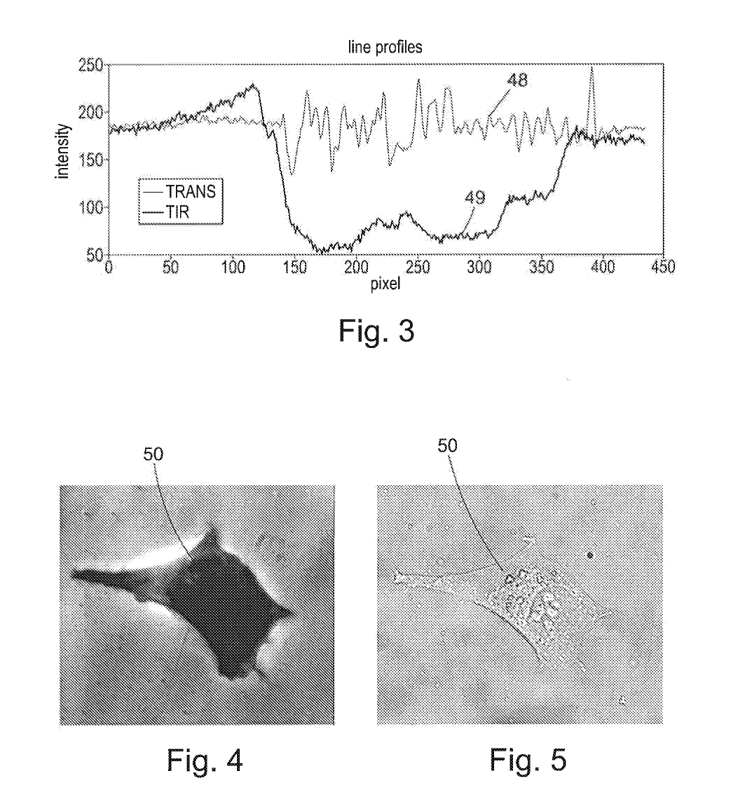

FIG. 3 is a graph comparing image contrast in FIG. 4 and FIG. 5;

FIG. 4 is an image of a cell in TIRM;

FIG. 5 is a bright field image of the cell shown in FIG. 4;

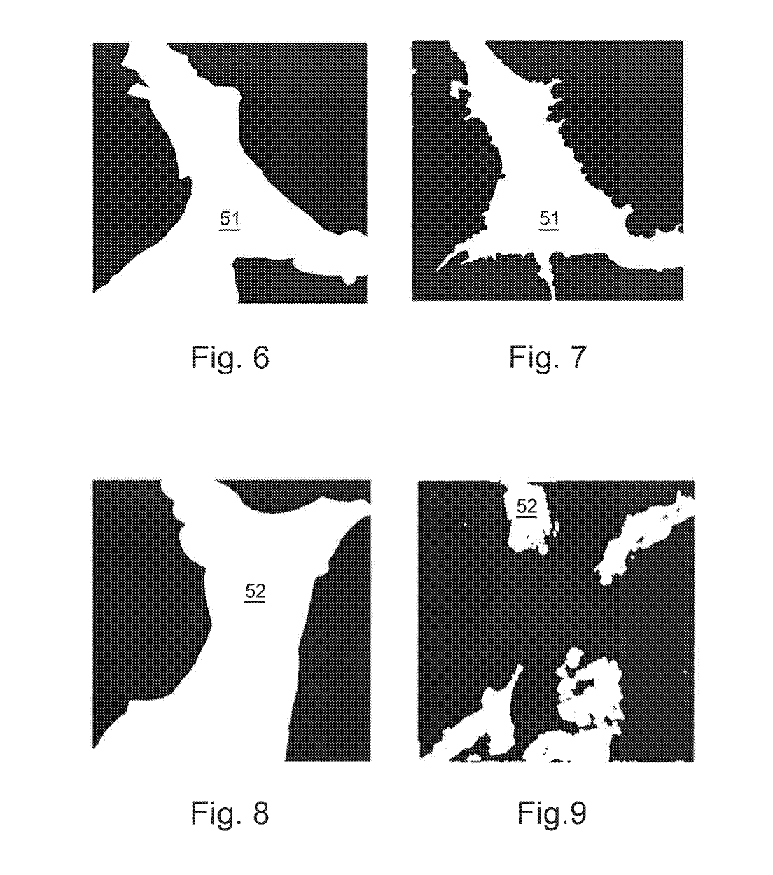

FIG. 6 is a bright field image of a cell that has been analysed with an image processing technique to distinguish the cell (shown in white) from the background (black);

FIG. 7 is a TIRM image of the cell shown in FIG. 6 and is also processed to distinguish the cell from the background;

FIG. 8 is a bright field image of a cell processed in the same way as the images in FIGS. 6 and 7;

FIG. 9 is a TIRM image of the cell shown in FIG. 8 and processed in the same way as FIGS. 6, 7 and 8;

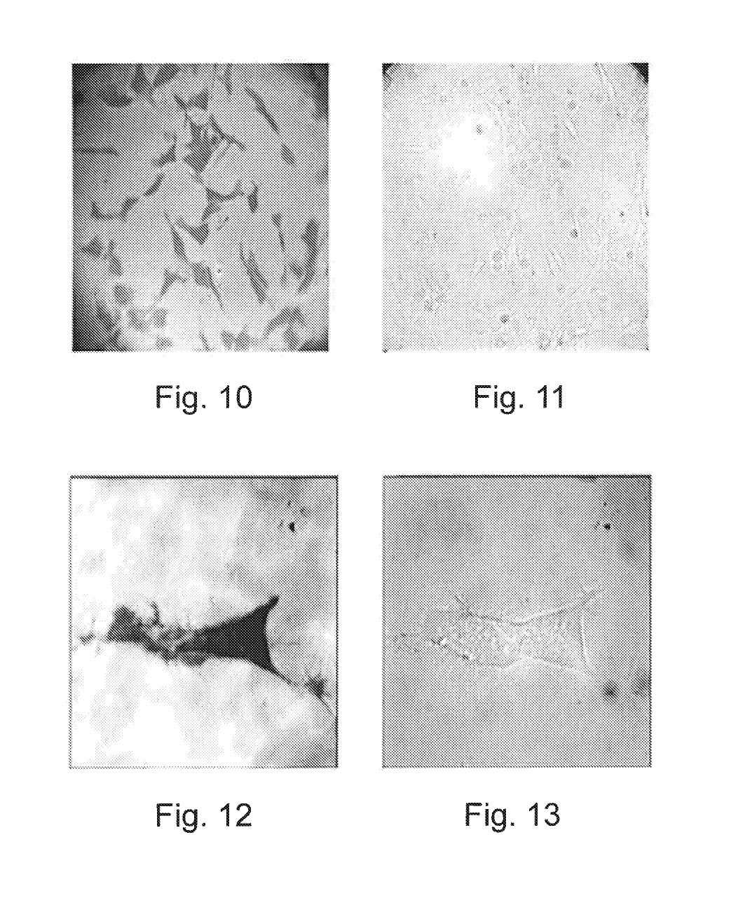

FIG. 10 is a TIRM image of a sample comprising a plurality of neural progenitor cells;

FIG. 11 is a bright field image corresponding to FIG. 10;

FIG. 12 is a TIRM image of the sample shown in FIG. 10, but with a smaller field of view;

FIG. 13 is a bright field image corresponding to FIG. 12;

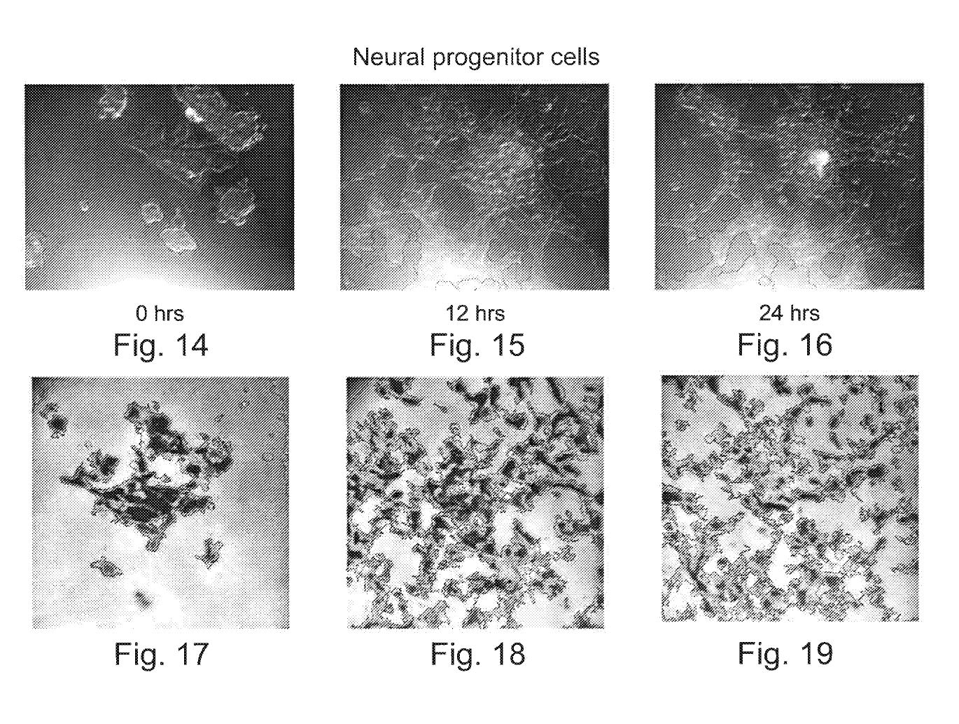

FIGS. 14, 15 and 16 are phase contrast images from a time course experiment, in which differentiation of neural progenitor cells to glial cells took place;

FIGS. 17, 18 and 19 are TIRM images, which correspond with the bright field images in FIGS. 14, 15 and 16 respectively;

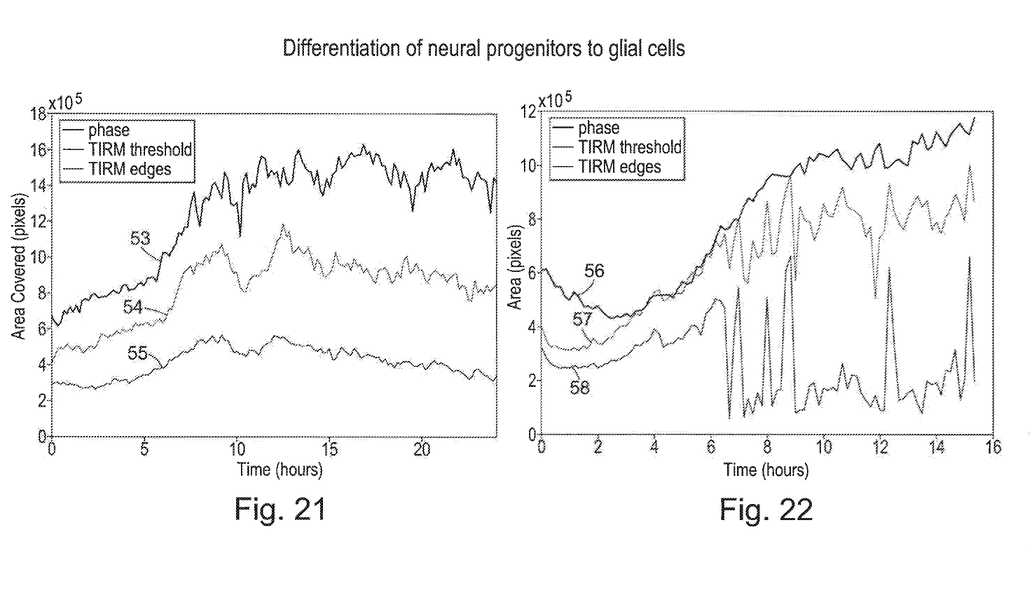

FIGS. 20 and 21 are graphs showing results from time course experiments;

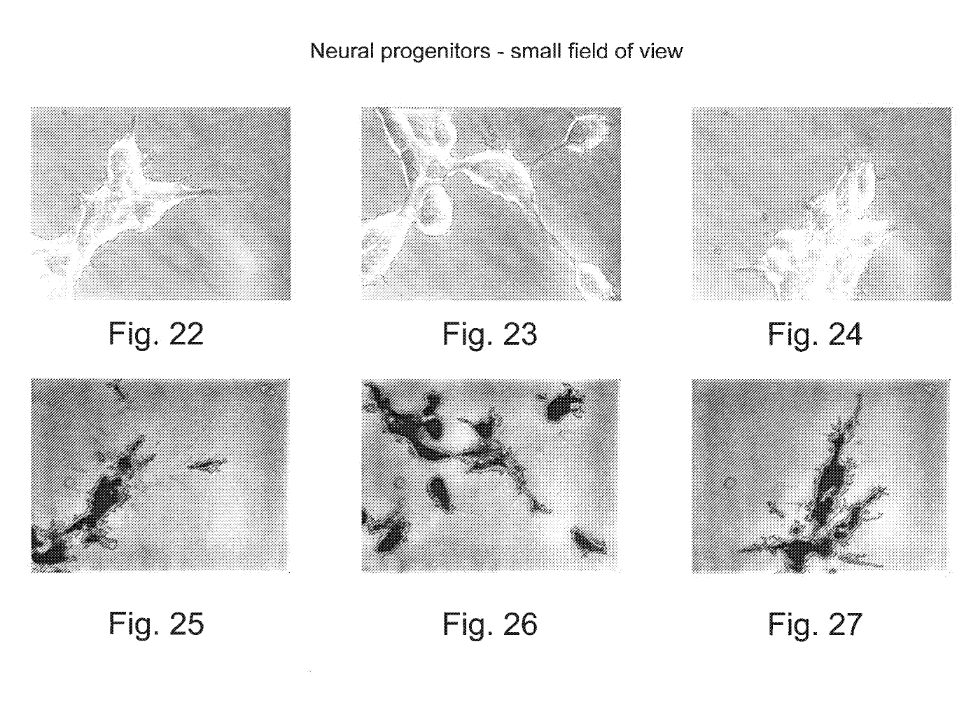

FIGS. 22, 23 and 24 are phase contrast images from a time course experiment, in which differentiation of neural progenitor cells to glial cells took place;

FIGS. 25, 26 and 27 are TIRM images, which correspond with FIGS. 22, 23 and 24 respectively;

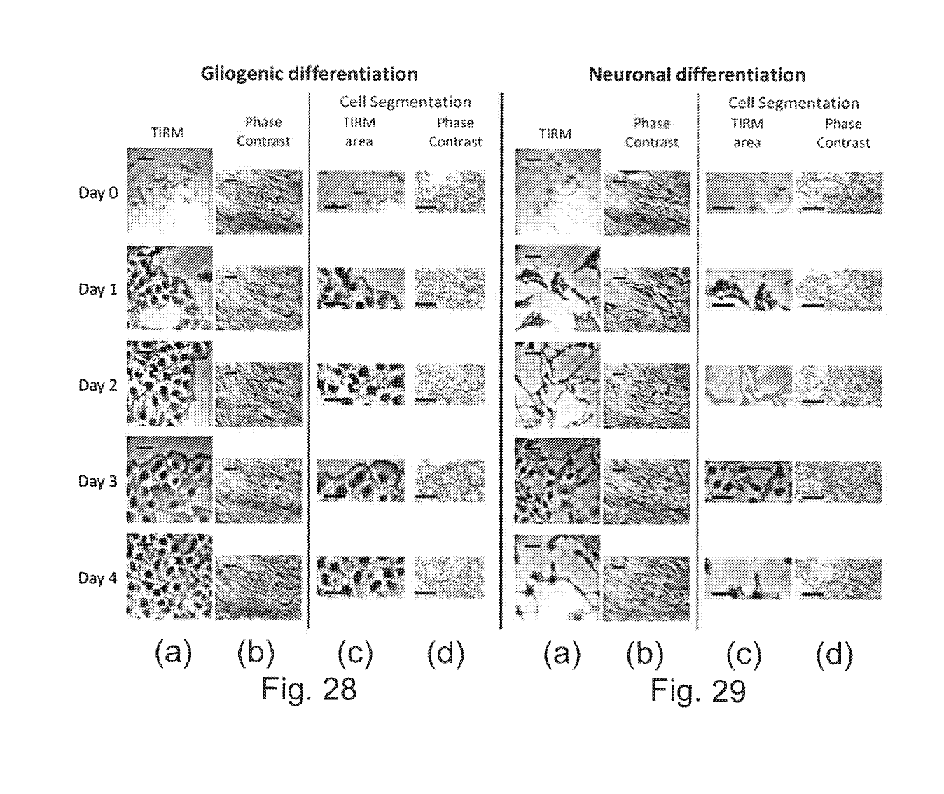

FIGS. 28 and 29 show TIRM and phase contrast images from a study in which adult mouse neural stem cells were differentiated towards a gliogenic and neurogenic fate;

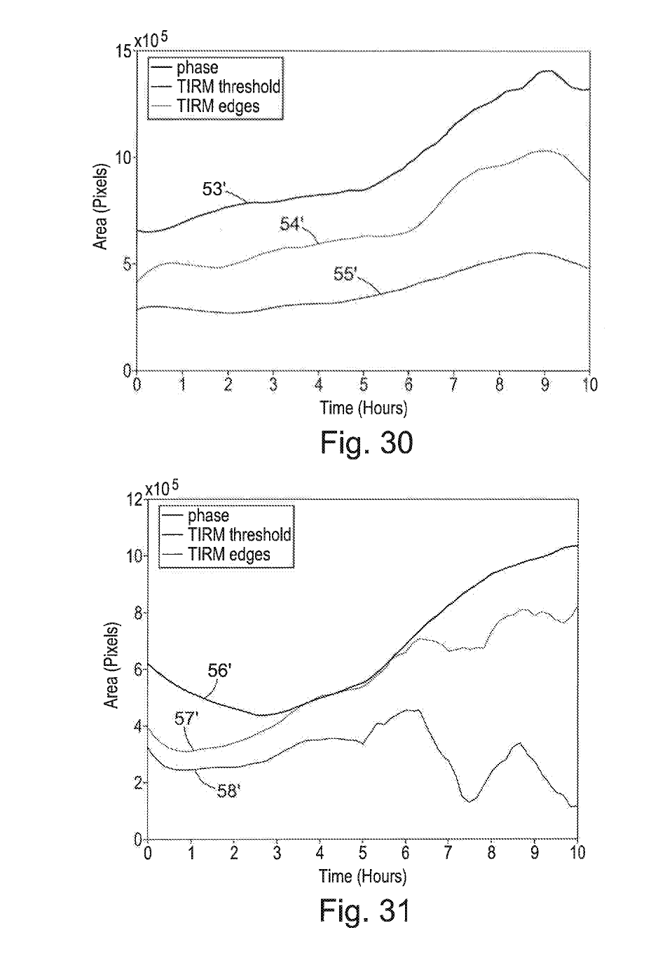

FIGS. 30 and 31 are graphs showing results from time course experiments;

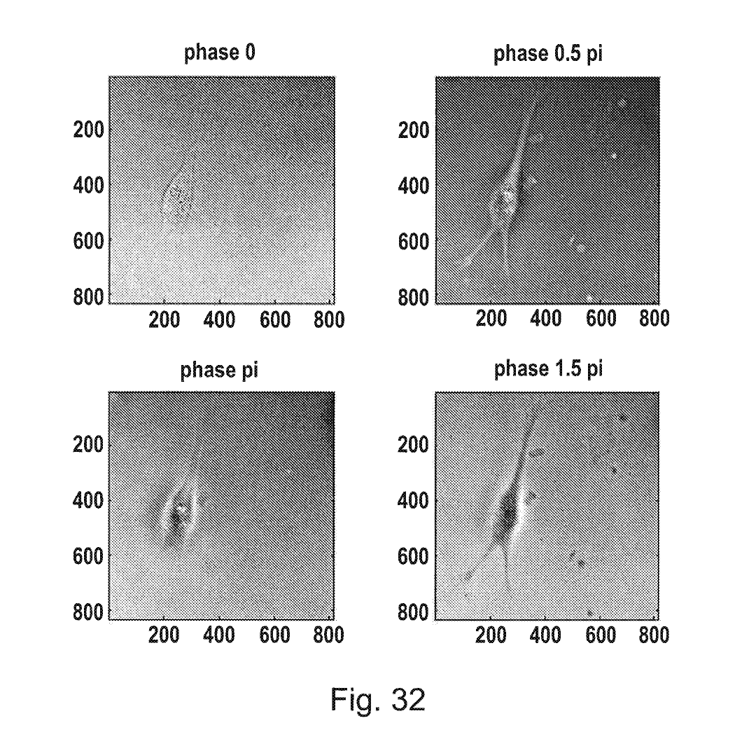

FIG. 32 shows four phase contrast images showing the results of phase stepping;

FIG. 33 is an image showing cell topography, which has been reconstructed from phase stepped images;

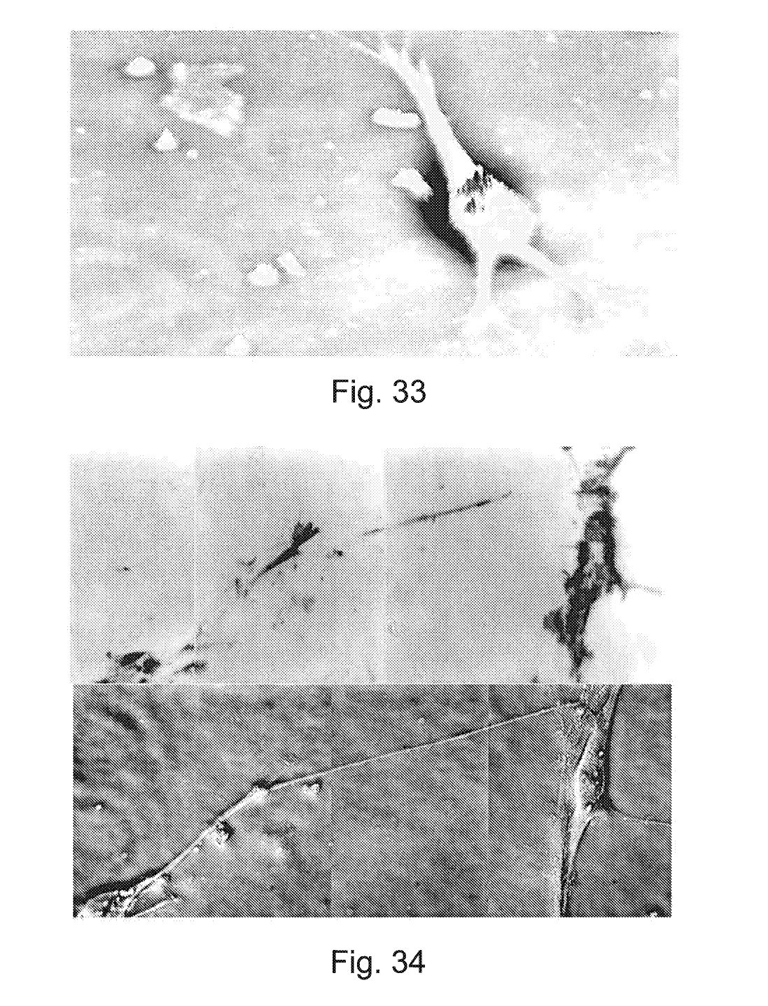

FIG. 34 shows TIRM and phase contrast images of neural progenitor cells, the images being formed from stitching together different fields of view; and

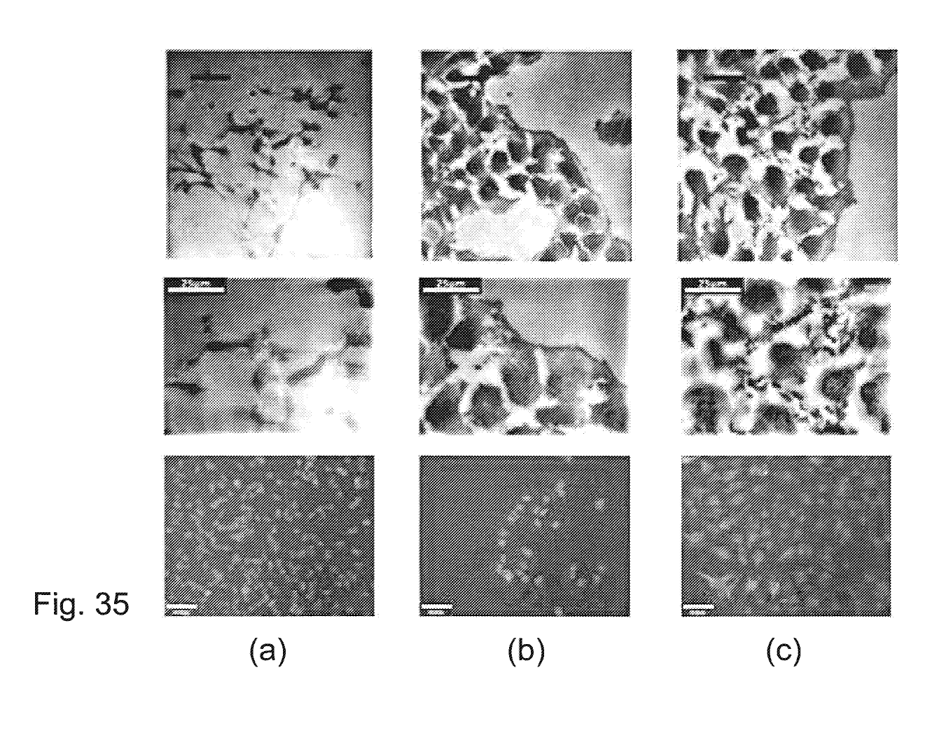

FIG. 35 compares TIRM images with marker expression of morphological changes associated with glial cells.

FIG. 1 shows a microscope 1 according to the invention set up on a laboratory bench 2. The microscope 1 is operable to image a sample using TIRM and phase contrast transmitted light microscopy.

FIG. 2 illustrates schematically how the microscope 1 works. The microscope 1 comprises a sample chamber 11. The conditions within the sample chamber 11 are controllable. Typically, the sample chamber 11 is an incubator. The conditions within the sample chamber 11 may, for instance, be controlled such that the temperature is 37° C. and/or the atmosphere contains 5% CO₂.

A sample 12 is located within the sample chamber 11. Typically, the sample may comprise a monolayer culture containing one or more cells.

The phase contrast capability of the microscope 1 will now be described with reference to FIG. 2. The microscope 1 comprises a first light source 13 comprising a blue LED. Light emitted from the first light source 13 passes through a first collector lens 14 and is then reflected by a first mirror 15 in the direction of a phase ring 16. After having passed through the phase ring 16, the light enters a first condenser 17, which collimates the light beam. The light beam then passes through the sample 12 within the sample chamber 11 and into an objective lens 18. Instead of a phase ring, the sample can be illuminated using a ring of light. The size and dimensions of the ring of light may be controllably variable.

An objective warmer 19 is provided around the objective lens 18. The objective warmer 19 is operable to control the temperature of the objective lens 18. The objective lens 18 is provided on a movable stage 20. The movable stage 20 can be moved in the x- and y- directions, thereby allowing viewing of different regions of the sample 12 and/or scanning of the sample 12. The movable stage 20 can also be moved in the z-direction, thereby enabling stacks of images to be obtained.

The objective lens 18 provides a magnification of ×60 and has a numerical aperture of 1.49.

After passing through the objective lens 18, the light beam passes through a first beam splitter 21 and a second beam splitter 22 without being split or redirected to a second mirror 23, which directs the light beam towards a phase plate 24 located in the back focal plane. After passing through the phase plate 24, a third beam splitter 25 separates the beam. A first part of the beam passes through a first imaging lens 27 and a first blue filter 29 before arriving at a first detector 44. A second part of the beam is reflected by a third mirror 26 towards a second imaging lens 28. The second part of the beam passes through the second imaging lens 28 and a second blue filter 30 before arriving at a second detector 45.

The principles of phase contrast microscopy are well understood. The optical system described above and shown in FIG. 2 is one example of an optical system for phase contrast microscopy.

In an embodiment, the phase plate can be replaced by a spatial light modulator to enable the size and contrast of the plate to be programmatically changed. By changing the contrast (phase of each pixel on the SLM), the microscope can obtain phase stepped images.

The TIRM capability of the microscope 1 will now be described with reference to FIG. 2. The microscope 1 comprises a second light source 31 comprising a red

LED. Light emitted from the second light source 31 passes through a second collector lens 32. The light then passes through a first projection lens 33, a TIRM annulus 34, a second projection lens 35 and a field aperture 36. After passing through the field aperture 36 the light passes through a second condenser 37, after which it is redirected in the first beam splitter 21 into the objective lens 18. The underside of the sample 12 is illuminated by the evanescent wave generated by total internal reflection of the light. The totally internally reflected light passes back through the objective lens 18. It then passes through the first beam splitter 21 without being redirected, before being redirected in the second beam splitter 22.

The light beam is then separated in a fourth beam splitter 38. A first part of the beam passes through a third imaging lens 41 and a first red filter 43 before arriving at a third detector 43. A second part of the beam is reflected by a fourth mirror 39 towards a fourth imaging lens 40. The second part of the beam passes through the fourth imaging lens 40 and a second red filter 42 before arriving at a fourth detector 46.

The principles of TIRM are well understood. The optical system described above and shown in FIG. 2 is one example of an optical system for TIRM.

The microscope 1 allows the simultaneous capture of phase contrast and TIRM images of the sample 12 in two fields of view. The microscope may be operable to provide continuous time-lapsed imaging.

The two LED light sources may be operable to emit the same wavelength of light. In some embodiments, the two LED light sources may emit red light. The use of red light may be preferred, since it has been found that cell viability can be lower following exposure to short wavelength, e.g. blue, light, as compared to long wavelength, e.g. red, light. When the two LED light sources emit the same wavelength of light, the microscope is operated so as to obtain images sequentially (rather than simultaneously) using each imaging modality. The light source for each imaging modality may be switched on and off, so that the light source for only one imaging modality is on at a given time.

Accordingly, in use, the LED light sources may be modulated individually.

In an embodiment, the microscope may comprise a spatial light modulator (SLM), which can be used as a programmable phase plate. Using a spatial light modulator as a programmable phase plate enables phase stepping and the acquisition of sequential, e.g. alternate, phase contrast and bright field images. Phase stepping can be used to obtain the actual phase and to reconstruct the topography of the biological or chemical material being imaged, e.g. cells.

The TIRM optical system may comprise a mask to block light with unwanted incident angles, thereby improving image contrast.

The microscope may comprise one or more cameras for capturing the images. Each camera may comprise a charge-coupled device (CCD) camera. In an embodiment, the microscope may comprise two CCD cameras arranged to obtain images with a wide and a small field of view simultaneously.

The microscope may comprise a movable stage or scanner associated with the objective lens for obtaining, in use, a stack of images in the z-plane.

FIG. 4 is an image of a cell 50 in TIRM. FIG. 5 is a bright field image of the cell 50. The image contrast in FIG. 4 is significantly greater than in FIG. 5. FIG. 3 is a graph which illustrates the difference in image contrast between FIG. 4 and FIG. 5. FIG. 3 shows line profiles, with intensity plotted on the y-axis and pixels plotted on the x-axis. A first line 48 shows the variation in intensity across the transmitted light bright field image (i.e. FIG. 5). A second line 49 shows the variation in intensity across the TIRM image (i.e. FIG. 4). The variation in intensity shown by the first line 48 and the second line 49 correspond with the difference in image contrast that can be observed between the transmitted light bright field image (FIG. 5) and the TIRM image (FIG. 4).

FIG. 6 is a bright field image of a cell 51. The image has been adjusted using image processing routines to separate the cell (white) from the background (black). FIG. 7 is an equivalent image of the cell 51 in TIRM, also processed. In FIG. 6, the cell 51 covers 39% of the area of the image. In FIG. 7, the cell 51 covers 31% of the image. TIRM has only a small penetration depth. Accordingly, TIRM only illuminates a thin region of the cell 51 above the glass substrate. Therefore, the difference in the area of the image taken up by the cell in FIG. 6 compared with in FIG. 7 may be due to regions of the cell that are not very close to, e.g. adhered to, the substrate. If the cell covered the same proportion of the image in FIG. 7 as in FIG. 6, then this might suggest that the whole of the underside of the cell was adhered to the substrate.

FIG. 8 is a processed image of a cell 52 in phase contrast. FIG. 9 is an equivalent image of the cell 52 in TIRM. In FIG. 8, the cell 52 covers 47% of the area of the image. In FIG. 9, the cell 52 covers 20% of the image. Accordingly, it can be inferred that only around 20% of the underside of the cell 52 is adhered to the substrate.

The cell 52 shown in FIGS. 8 and 9 is relatively poorly adhered to the substrate in comparison with the cell 51 shown in FIGS. 6 and 7.

An assessment of cell adhesion may be made by comparing a TIRM image with an equivalent image produced using bright field or phase contrast light microscopy. Cell adhesion is tightly linked to cell functionality and morphology. Accordingly, measuring cell adhesion can be a useful way to monitor and/or characterise cells.

Comparison of the images may be used to produce a parameter or a metric for characterising cells. This may be done automatically using image processing software.

For instance, a ratio of the cell area adhered to the substrate to the cell area not adhered to the substrate may be a useful metric or parameter.

FIGS. 10, 11, 12 and 13 are images of a sample in TIRM (FIGS. 10 and 12) and bright field imaging (FIGS. 11 and 13). The sample contains neural progenitor cells. FIGS. 10 and 11 have a larger field of view and FIGS. 12 and 13 have a smaller field of view. There is a clear difference in image contrast between the TIRM images (FIGS. 10 and 12) and the bright field images (FIGS. 11 and 13).

Bright field imaging may be used as an alternative to phase contrast imaging. Hence, comparison of bright field images with TIRM images may be used to produce a parameter of metric for characterising cells.

FIGS. 14, 15, 16, 17, 18 and 19 illustrate a time course experiment, in which differentiation of neural progenitor cells to glial cells takes place.

FIG. 14 is a phase contrast image of the sample containing the cells at the start of the experiment. FIG. 17 is an equivalent TIRM image to FIG. 14.

FIG. 15 is a phase contrast image of the sample containing the cells after 12 hours of the experiment. FIG. 18 is an equivalent TIRM image to FIG. 15.

FIG. 16 is a phase contrast image of the sample containing the cells after 24 hours of the experiment. FIG. 19 is an equivalent TIRM image to FIG. 16.

FIG. 20 is a graph of a time course experiment of the kind illustrated in FIGS. 14, 15, 16, 17, 18 and 19. Area covered, measured in pixels, is plotted on the y-axis and time, measured in hours, is plotted on the x-axis. A line 53 represents the area covered, as measured from phase contrast images. A line 55 represents the area covered, as measured from TIRM threshold. A line 54 represents the area covered, as measured by TIRM edges.

The lines 54, 55 for TIRM are below the line for phase contrast 53. This is as expected, since the cross-section of the cells seen in phase contrast may be larger than the surface area of the cells that is adhered to the substrate, as seen in TIRM.

FIG. 21 is a similar graph to that of FIG. 20. Again, area covered, measured in pixels, is plotted on the y-axis and time, measured in hours, is plotted on the x-axis.

A line 56 represents the area covered, as measured from phase contrast images. A line 58 represents the area covered, as measured from TIRM threshold. A line 57 represents the area covered, as measured by TIRM edges.

As in FIG. 20, the lines 57, 58 for TIRM are generally below the line 56 for phase contrast. However, lines 56 and 57 converge after around three hours of the experiment. The two lines 56, 57 are almost on top of each other from around three hours to six hours into the experiment. It is thought that the convergence of the lines may indicate the onset of cell differentiation.

FIGS. 22, 23, 24, 25, 26 and 27 illustrate a time course experiment, in which differentiation of neural progenitor cells to glial cells takes place. The field of view is relatively small compared with the images shown in FIGS. 14, 15, 16, 17, 18 and 19.

FIG. 22 is a phase contrast image of the sample containing the cells at the start of the experiment. FIG. 25 is an equivalent TIRM image to FIG. 22.

FIG. 23 is a phase contrast image of the sample containing the cells after 12 hours of the experiment. FIG. 26 is an equivalent TIRM image to FIG. 23.

FIG. 24 is a phase contrast image of the sample containing the cells after 24 hours of the experiment. FIG. 27 is an equivalent TIRM image to FIG. 24.

In an experiment, adult mouse neural stem cells were cultured and differentiated towards a gliogenic and a neurogenic fate. The attachment profile was monitored using TIRM and corresponding phase contrast images across a four-day differentiation period. The images were segmented to identify attachment area and are covered with cells in the phase contrast images.

FIG. 28 shows images of adult mouse neural stem cells that were differentiated towards a gliogenic fate. The top row of four images shows the cell culture at the start of the experiment. The second row down shows the cell culture after one day, the third row down shows the cell culture after two days, the fourth row down shows the cell culture after three days and the fifth row down shows the culture after four days. Columns (a) and (c) show TIRM images; each image in column (c) is segmented from its corresponding image in column (a). Columns (b) and (d) show phase contrast images; each image in column (d) is segmented from its corresponding image in column (b).

FIG. 29 shows images of adult mouse neural stem cells that were differentiated towards a neurogenic fate (neuronal differentiation). The top row of four images shows the cell culture at the start of the experiment. The second row down shows the cell culture after one day, the third row down shows the cell culture after two days, the fourth row down shows the cell culture after three days and the fifth row down shows the culture after four days. Columns (a) and (c) show TIRM images; each image in column (c) is segmented from its corresponding image in column (a). Columns (b) and (d) show phase contrast images; each image in column (d) is segmented from its corresponding image in column (b).

The scale bars shown in FIGS. 28 and 29 represent 50 micrometres.

The segmented TIRM images in column (c) in FIGS. 28 and 29 were used to identify the attachment area, i.e. the area of cell adhered to the substrate. The segmented phase contrast images in column (d) in FIGS. 28 and 29 were used to identify the overall cross-section of the cells.

As can be seen from FIGS. 28 and 29, neural progenitors (Day 0) and cells exposed to cell culture conditions promoting gliogenic (FIG. 28, Day 1, Day 2, Day 3, Day 4) and neurogenic differentiation (FIG. 29, Day 1, Day 2, Day 3, Day 4) have different attachment profiles depending on culture conditions.

FIG. 30 is a graph of the first 10 hours of the time course experiment plotted in FIG. 20. Area covered, measured in pixels, is plotted on the y-axis and time, measured in hours, is plotted on the x-axis. A line 53′ represents the area covered, as measured from phase contrast images. A line 55′ represents the area covered, as measured from TIRM threshold. A line 54′ represents the area covered, as measured by TIRM edges.

The lines 54′, 55′ for TIRM are below the line for phase contrast 53′. This is as expected, since the cross-section of the cells seen in phase contrast may be larger than the surface area of the cells that is adhered to the substrate, as seen in TIRM.

FIG. 31 is a similar graph to that of FIG. 30. Again, area covered, measured in pixels, is plotted on the y-axis and time, measured in hours, is plotted on the x-axis. A line 56′ represents the area covered, as measured from phase contrast images. A line 58′ represents the area covered, as measured from TIRM threshold. A line 57′ represents the area covered, as measured by TIRM edges.

As in FIG. 30, the lines 57′, 58′ for TIRM are generally below the line 56′ for phase contrast. However, lines 56′ and 57′ converge after around three hours of the experiment. The two lines 56′, 57′ are almost on top of each other from around three hours to six hours into the experiment. It is thought that the convergence of the lines may indicate the onset of cell differentiation.

FIG. 32 shows four phase contrast images of a cell, the images being produced by phase stepping. In the top left image, the phase was 0. In the top right image, the phase was 0.5 π. In the bottom left image, the phase was 7E. In the bottom right image, the phase was 1.5 π.

FIG. 33 is an image showing cell topography, which has been reconstructed from phase stepped images. Phase stepped images can be obtained through use of a spatial light modulator.

The top half of FIG. 34 shows a TIRM image of neural progenitors, the image having been formed by stitching together different fields of view. The bottom half of

FIG. 34 shows a corresponding phase contrast image of the neural progenitors, the image having been formed by stitching together different fields of view.

FIG. 35 contains nine images arranged in three columns, indicated as (a), (b) and (c). The top two rows include TIRM images. The bottom row includes transmitted light microscopy images in which cell nuclei are shown by DAPI marker expression. The images record a time course study and allow for a comparison of TIRM with marker expression. The TIRM images and the expression of the DAPI markers may provide complementary information relating to cell differentiation. In column (a), at day 0 of the time course study, neural progenitors are present with cell nuclei shown in purple by DAPI marker expression. In column (b), after 1 day, morphological changes associated with glial cells can be seen in the TIRM images. However, the cell nuclei are still shown in purple by DAPI marker expression. In the bottom image of column (c), after two days, GFAP marker expression seen in red can be seen, which is indicative of glial cell type.

Advantageously, the method and microscope of the invention may have a predictive capability. Results obtained from a time course study of directed differentiation of neural progenitor cells carried out by the applicant indicate that TIRM images can predict the onset of differentiation earlier than immuno-staining.

As noted above, regenerative medicine applications include, among others the production of cellular products and cell therapies. For these it is vital to monitor and characterise cells throughout the manufacturing process. Currently used systems often involve the sacrifice of cell populations for endpoint measurements and/ or the usage of fluorescence labels which are associated with phototoxicity. Label free microscopy may provide a means to circumvent these problems and enable real-time live cell imaging during manufacturing processes or to monitor differentiation events.

Advantageously, the invention provides a label-free imaging technique which enables monitoring and characterisation of live cells in real time. Utilising total internal reflection microscopy in combination with reflected or transmitted light microscopy, e.g. using phase contrast illumination, bright field illumination, cross-polarized light illumination or dark field illumination, may allow the generation of a “cellular fingerprint”. Comparing these areas of cell surface attachment, as shown by the total internal reflection images, to whole cell area, indicated, for example, in phase contrast images, exemplifies one of the unique image parameters that can be obtained to study cell health and cell type.

In an embodiment, the invention has been utilised to monitor neural differentiation events. Adult mouse neural stem cells were subjected to established neurogenic and gliogenic differentiation culture conditions. In parallel to the image analysis carried out in accordance with the invention, successful differentiation was corroborated by endpoint validation techniques such as flow cytometry, immunofluorescence and qRT-PCR.

The combination of total internal reflection microscopy and reflected or transmitted light microscopy enables label-free real-time live cell imaging of adherent in vitro cell culture systems. Furthermore, it may provide a platform with which cell parameters can be extracted which allow differentiation events to be studied in real-time. In addition, the invention can be used to extract cell quality parameters for future application of online monitoring of cells in manufacturing processes for clinical applications to aid in process optimisation and quality control.

The invention realises the advantages of label-free imaging, which may include: being able to image live cells over a period of time; having no need to use fluorescent labels, which may lead to problems with phototoxicity; being non-destructive and not requiring the sacrifice of a portion of the material of interest.

Potential applications of the invention may include: live cell imaging for studying fundamental cell biology; characterisation of cells under different cell culture conditions; and monitoring the quality of cellular products for therapeutic purposes.

It is envisaged that the invention may allow for long-term continuous monitoring of differentiation events in real time. A combination of biomarker analysis with TIRM may be used to validate differentiation events.

It is also envisaged that the invention may be used to monitor cell attachment to different surfaces and biomaterials.

Image processing and/or analysis may allow for the acquisition of footprints of different cell types and the extrapolation of parameters to identify different cell types.

Advantageously, the invention may identify parameters to monitor cell health.

1. A method of monitoring and/or characterising biological or chemical material arranged on a substrate comprising:
producing a first image of the biological or chemical material using total internal reflection microscopy (TIRM); and
producing a second image of the biological or chemical material using transmitted or reflected light microscopy.

2. A method according to claim 1, wherein the biological or chemical material comprises one or more bacteria or cells.

3. A method according to claim 2, wherein the cell(s) are provided in a culture comprising the cell(s) and a growth medium.

4. A method according to claim 3, wherein the cell(s) are provided in a monolayer culture.

5. A method according to claim 1, wherein the second image is produced using phase contrast illumination, bright field illumination, cross-polarized light illumination or dark field illumination.

6. A method according to claim 1, wherein the first image and the second image are produced substantially simultaneously or sequentially.

7. A method according to claim 1, wherein the first image and the second image are of substantially the same portion of the biological or chemical material.

8. A method according to claim 1 further comprising processing and/or analysing the first image and the second image.

9. A method according to claim 8, wherein processing and/or analysing the first image and the second image comprises comparing the first image with the second image.

10. A method according to claim 8, wherein the image processing and/or analysis generates a parameter or a metric.

11. A method according to claim 10, wherein processing and/or analysing the first image and the second image comprising comparing one or more parameters or metrics derived from the first image and the second image.

12. A method according to claim 10, wherein the parameter or metric comprises a ratio of the surface area of the biological or chemical material that appears to be adhered to the substrate to the overall surface area or cross section of the biological or chemical material.

13. A method according to claim 1, wherein the chemical or biological material arranged on the substrate is located within a chamber, e.g. an incubator, operable to provide a controlled environment.

14. A method according to claim 1, wherein the method is carried out over a period of time, in order to monitor the chemical or biological material over the period of time.

15. A method according to claim 14, wherein the method is used to monitor cell differentiation or the response of cells or bacteria to a stimulus such as a chemical agent, a disease, a drug or a change in conditions.

16. A microscope for monitoring and/or characterising biological or chemical material on a substrate comprising:
a first optical system for viewing and/or imaging the biological or chemical material using total internal reflection microscopy; and
a second optical system for viewing and/or imaging the biological or chemical material using reflected or transmitted light microscopy;
wherein the first optical system and the second optical system share an objective lens.

17. A microscope according to claim 16, wherein the shared objective lens has a numerical aperture of 1.4 or more.

18. A microscope according to claim 16, wherein the shared objective lens provides a magnification of at least 20×.

19. A microscope according to claim 16, comprising a movable stage associated with the shared objective lens.

20. A microscope according to claim 16, wherein the first optical system and/or the second optical system comprises a monochromatic source of light.

21. A microscope according to claim 16 further comprising image processing and/or analysis means, operable to process and/or analyse images produced by the first optical system and/or the second optical system.

22. A microscope according to claim 16 comprising a spatial light modulator.

23. A microscope according to claim 16, wherein the first optical system and/or the second optical system is provided with one or more image capture devices.

24. A microscope according to claim 16, which is part of or is attached or attachable to an apparatus for manufacturing or producing cells, e.g. for use in regenerative medicine.

25. A microscope according to claim 16, wherein the microscope is portable and/or is an add-on for an existing commercial microscope.

26. A method of manufacture of biological material, e.g. cells, comprising:
culturing or growing the biological material; and
characterising and/or monitoring the biological material by a method according to claim 1.

27. A method of manufacture of biological material, e.g. cells, comprising:
culturing or growing the biological material; and
using a microscope according to claim 16 to characterise and/or monitor the biological material.
